(19) United States
(12) Patent Application Publication
(10) Pub. No.: US 2014/0157612 A1
(43) Pub. Date: Jun. 12, 2014

(54) HAND DRYER

(71) Applicant: Liz Johanna Angarita Duran, Alicante (ES)

(72) Inventor: Liz Johanna Angarita Duran, Alicante (ES)

(21) Appl. No.: 14/102,925

(22) Filed: Dec. 11, 2013

(30) Foreign Application Priority Data
Dec. 11, 2012 (ES) 201231926

(51) Int. Cl. A61L 2/18; A61L 9/03

(52) U.S. Cl. CPC A61L 2/18; A61L 9/037
USPC 34/60

(57) ABSTRACT

The present invention refers to a hand dryer which, in addition to drying the hands by means of a warm air exit, also propels a sprayed germicidal or bactericidal liquid, or any other personal hygiene product. Additionally, the dryer incorporates an air freshener for emitting scents. In order to do so, it comprises a housing (1) with a separation wall (2) which separates the elements participating in the hand dryer and an isolated and independent zone in which a scent evaporation air freshener (4) participates; where a duct coming from a germicidal or bactericidal liquid container (7) ends in the warm air exit zone. In the zone where the evaporation air freshener is established, a heating element (18) for evaporating the scent is established.

### OBJECT OF THE INVENTION

The present invention refers to a hand dryer which, in addition to drying the hands by means of a warm air exit, also propels a sprayed germicidal or bactericidal liquid, or any other personal hygiene product. Additionally, the dryer incorporates an air freshener for emitting scents as a part of the housing thereof.

It is thus an object of the invention to provide a hand dryer which also carries out hygienic functions and which also allows for establishing an evaporation air freshener forming part of the dryer assembly.

### BACKGROUND OF THE INVENTION

It is known that hand dryers that function by propelling warm air are based on actuating a turbine propelling air through an electrical resistor, whereby the air is heated and exits through a port or nozzle under which the hands to be dried are placed.

These hand dryers do not perform any function other than drying the hands by means of warm air, not providing any hygienic function of the hands to be dried or of course emitting a scent by evaporation, as the dryer which is the object of the present invention.

### DESCRIPTION OF THE INVENTION

The hand dryer is formed by a housing having two parts which are completely independent and isolated therebetween, one corresponding to the elements participating in the air propulsion, heating thereof and suitable control and actuation means, while the other part which is isolated and independent from the first part includes a container of a germicidal or bactericidal liquid which allows for the introduction of said liquid towards the warm air exit zone by means of a duct having solenoid valve suitable controlled by means of an electronic circuit, thereby causing the emission of liquid molecules, and where the liquid in the germicidal container is sucked by the Venturi effect that appears in the warm air exit zone.

Therefore, in the first place the hand dryer not only carries out a drying function by means of warm air, but it also sprays liquid molecules of a germicidal bactericidal product contained in a container which is isolated and independent with respect to the plurality of components participating in the hand dryer itself.

Additionally, the part of the housing which is independent from the hand dryer, where the germicidal bactericidal container is located, includes an air freshener formed by a deposit of scent mounted on a support, having an outside air access and a wick absorbing the liquid scent from the deposit, and by means of a heating element controlled by a regulation potentiometer, the aromatic liquid is evaporated and exits towards the outside of the air freshener through a grille located in the upper part, therefore the dryer, in addition to the two functions disclosed above, also fulfils an air freshener function by means of the evaporation of an aromatic liquid.

In an alternative embodiment, the duct communicating the germicidal container to the hand dryer exit zone may end directly in said zone or else in a narrow internal perimeter area having holes for obtaining a more homogeneous air-liquid mix.

### DESCRIPTION OF THE DRAWINGS

In order to complement the description below and with the object to aid in a better understanding of the features of the invention, according to an example of a preferred embodiment thereof, a set of drawings being an integral part of said description is attached where, by way of illustration and not of limitation, the following is represented:

FIG. 1 Shows a lateral view of the hand dryer which is the object of the invention with the part showing the separation of the dryer itself with respect to the zone where the air freshener is established.

FIG. 2 Shows an schematic view of the inside corresponding to the hand dryer and air freshener assembly represented in the above figure, where all the components participating in said assembly are visible.

FIG. 3 Shows an schematic view of an alternative embodiment where the duct coming from the germicidal or bactericidal liquid container ends in a narrow inside perimeter zone of the warm air exit port corresponding to the hand dryer itself.

### PREFERRED EMBODIMENT OF THE INVENTION

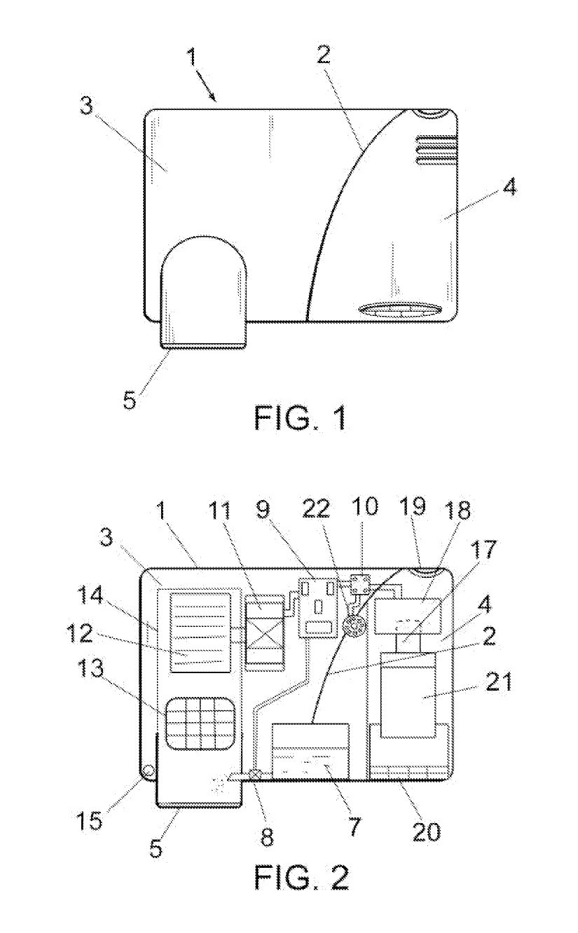

As shown in the above figures, and specifically in connection with FIGS. 1 and 2, the hand dryer is formed by a housing (1) having a wall (2) for separating and isolating zone (3) corresponding to the hand dryer itself from zone (4) corresponding to an evaporation air freshener.

As to the hand dryer itself, it includes a port (5) for the exit of warm air, close to which a duct (6) coming from a germicidal or bactericidal liquid container (7) ends, said duct (6) having a closure solenoid valve (8) whose opening time is controllable by means of an electronic circuit (9) having its respective power supply (10), such that through said electronic circuit (9) a motor (11) is activated, said motor in turn making a turbine (12) turn for propelling air towards the exit port (5), said air having previously passed through a resistor (13) which heats the air propelled by the turbine (12), which is housed inside an internal housing (14), as shown in FIG. 2.

This way, air propelled by the turbine (12), after passing through the resistor (13), is heated up and exits through port (5), drying hands placed under said exit port (5), while the high pressure created in the exit zone causes the liquid from the duct (6) coming from the container (7) to be sucked by the Venturi effect, thereby producing liquid molecules which exit through the port (5), thus drying the hands and at the same time carrying out a hygienic cleaning thereof.

As in other devices of this type, an optical sensor (15) which activates when the hands are placed under the exit port (5) is included.

The air freshener (4) is placed at the other side of the separation and isolation wall (2), and it is formed by a deposit (16) which contains the freshening product which is sucked by means of a wick (17) in order to be evaporated by means of a heating element (18) and subsequently to cause the scent to exit through an upper grille (19), this assembly also having an air input (20) and a support (21) for the scent deposit (16).

A potentiometer or switch having several positions (22) allows for regulating the intensity through the resistors corresponding to the heating element (18), and therefore for regulating the evaporation capacity of the air freshener itself.

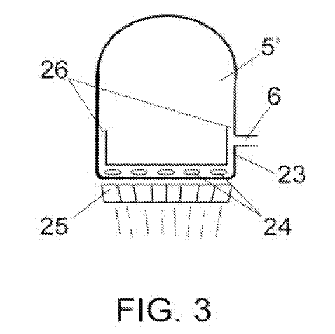

FIG. 3 shows an alternative embodiment for the positioning or the way the duct (6) coming from the germicidal or bactericidal liquid deposit (7) ends at the exit zone (5′) of the air freshener, such that in the case of the alternative embodiment shown in FIG. 3 said duct (6) ends in a narrow zone (23) through which pressurized air produces the exit of liquid molecules through the holes (24) established in the lower part or in an exit nozzle (25), as shown schematically in FIG. 3, where the arrows (26) indicate the pressurized air input towards the narrow (23) zones where the duct (6) from the germicidal bactericidal container (7) ends.

1. Hand dryer which includes a turbine (12) which is actuatable ay a motor (11) for propelling air which is heated by means of a resistor placed between the turbine itself and an exit port under which the hands to be dried are placed, being characterized by comprising a housing (1) which a separation wall (2) which separates the elements participating in the hand dryer and an isolated and independent zone in which a scent evaporation air freshener (4) is placed; where a duct coming from a germicidal or bactericidal liquid container (7) ends in the warm air exit zone, such that by means of the Venturi effect caused by the pressure, the duct is sucked and consequently the germicidal bactericidal liquid accesses the exit zone where the pressure of the warm air causes spraying towards the exit port; where said germicidal bactericidal liquid duct includes a solenoid valve (8) whose closure is controlled by means of the electronic circuit (9) controlling the elements participating in the dryer; where in the zone where the evaporation air freshener is established, the germicidal bactericidal liquid container is also located, said air freshener including a scent deposit which, by means of a wick (17), reaches a heating element (18) for evaporating the scent, which exits outside through an upper grille (19), while there is a lower outside air entrance grille (20).

2. Hand dryer according to claim 1, characterized in that the exit duct for the germicidal bactericidal liquid in the container has access to the dryer exit zone through a narrow zone having a pressurized air input, the air exiting outside through holes or through a lower nozzle, in order to achieve the spraying of the germicidal bactericidal liquid and its exit towards the hands for performing the hygienic cleaning function.
